(19) United States
(12) Patent Application Publication
(10) Pub. No.: US 2010/0312018 A1
(43) Pub. Date: Dec. 9, 2010

(54) Processes for Producing Levosandal and Levosandol

(76) Inventors: Avelino Corma Canos, Valencia (ES); Sara Iborra Chornet, Valencia (ES); Alexandra Velty, Valencia (ES)

Correspondence Address: INTERNATIONAL FLAVORS & FRAGRANCES INC., 521 WEST 57TH ST, NEW YORK, NY, 10019, US

(21) Appl. No.: 12/753,279

(22) Filed: Apr. 2, 2010

(30) Foreign Application Priority Data
Apr. 2, 2009 (EP) 09382045.4
Mar. 12, 2010 (EP) 10156421.9

(51) Int. Cl. C07C 45/74; C07C 29/159

(52) U.S. Cl. 568/446; 568/838

(57) ABSTRACT

The present invention relates to processes for producing 2-ethyl-4-(2,2,3-trimethyl-3-cyclopenten-1-yl)-2-butenal and 2-ethyl-4-(2,2,3-trimethyl-3-cyclopenten-1-yl)-2-buten-1-ol using heterogeneous bifunctional catalysts with a good yield. There is provided a process for producing 2-ethyl-4-(2,2,3-trimethyl-3-cyclopenten-1-yl)-2-butenal by the cross-aldol condensation between campholenic aldehyde and butanal using bifunctional heterogeneous catalysts in the presence of controlled amounts of an aliphatic alcohol; and a process for producing 2-ethyl-4-(2,2,3-trimethyl-3-cyclopenten-1-yl)-2-buten-1-ol useful as perfume, starting from 2-ethyl-4-(2,2,3-trimethyl-3-cyclopenten-1-yl)-2-butenal through a MPV reduction using an acid-base bifunctional heterogeneous catalyst. Both process can be coupled in a cascade process which involves the cross-aldol condensation between campholenic aldehyde and butanal followed by the Meerwein-Ponndorf-Verley (MPV) reduction in the presence of a secondary alcohol using the same heterogeneous bifunctional catalyst for obtaining (2-ethyl-4-(2,2,3-trimethyl-3-cyclopenten-1-yl)-2-buten-1-ol).

### CROSS REFERENCE TO RELATED APPLICATIONS

This application claims benefit to European Patent Application No. 10156421.9 filed on Mar. 12, 2010 and claims priority to European Patent Application No. 09382045.4 filed on Apr. 2, 2009.

### FIELD OF THE INVENTION

The present invention relates to processes for producing Levosandal (2-ethyl-4-(2,2,3-trimethyl-3-cyclopenten-1-yl)-2-butenal) and Levosandol (2-ethyl-4-(2,2,3-trimethyl-3-cyclopenten-1-yl)-2-buten-1-ol) using heterogeneous bifunctional catalysts.

### BACKGROUND OF THE INVENTION

α,β-unsaturated aldehyde compounds as well as α,β-unsaturated alcohols are useful compounds as perfumes, perfume precursors or intermediates for pharmaceuticals and agricultural chemicals.

One of the compounds which refer the present invention 2-ethyl-4-(2,2,3-trimethyl-3-cyclopenten-1-yl)-2-butenal is produced by the cross aldol condensation between campholenal and butanal in the presence of know homogeneous and heterogeneous aldol condensation catalysts.

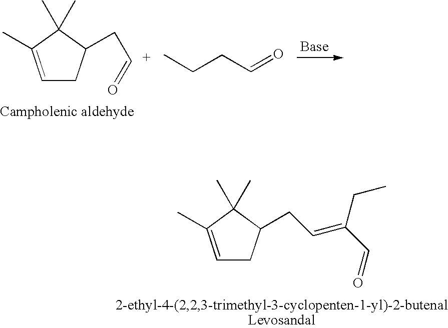

In the U.S. Pat. No. 4,318,831 (1982) Klein et al. describe a method for producing cyclopentene derivatives with application to the perfume preparation. Among the examples it is described the preparation of compounds by cross-aldol condensation between campholenal and aldehydes such as propanaldehyde and butyraldehyde using sodium ethoxyde as catalyst (10 wt % with respect to campholenal) at 0° C. The molar ratio aldehyde/campholenal is 2 and the aldehyde is dropped to campholenal to gradually react with each other. The neutralization of the catalysts is carried out with acetic acid and after distillation of the reaction mixture the α,β-unsaturated aldehyde is obtained in 60-80% yield. In a more recent patent, Ishida et al. (WO 2002/063703, 2007) describes a process for preparing α,β-unsaturated aldehyde compounds, among them 2-ethyl-4-(2,2,3-trimethyl-3-cyclopenten-1-yl)-2-butenal, by the cross-aldol condensation between camphonelal and butanal in the presence of an amine and a protonic acid having 4 to 20 carbons atoms or a salt thereof. The catalyst is neutralized at the end of the reaction using acetic acid. In general, the use of homogeneous catalysts involves the necessity of a neutralization step and the requirement of a thorough wash with water of the final reaction mixtures. Moreover, in some cases, self-condensation of the aldehydes involved also occurs, giving by-products which have to be eliminated by distillation. Besides, the distillation process leads to isomerization and polymerization reactions of the desired compounds decreasing the final yield. In order to overcome these inconveniencies, the use of heterogeneous catalysts appears as an interesting alternative for the production of α,β-unsaturated aldehydes. The patent WO 01/38278 (2001) describes a process for preparing α,β-unsaturated carbonyl compounds (among them 2-ethyl-4-(2,2,3-trimethyl-3-cyclopenten-1-yl)-2-butenal) using heterogeneous catalysts having basic characteristics. The catalysts able to perform the condensation between campholenal and butanal are among alkaline earth oxides, layered double hydroxides (LDH) of divalent and trivalent metals in their calcined form (mixed oxides) and hydrated form of these mixed oxides.

Levosandol is produced by reduction of the aldehyde precursor compound by different methods. For instance, in the patents WO2002022526 (2002) and EP 829463 (1998) are described the selective reduction of carbonyl compounds through a hydrogenation process using ruthenium phosphine complexes and where Levosandol is prepared by hydrogenation of the aldehyde precursor compound. In another patent the hydrogenation is performed using a Cu chromite catalyst (WO 9310068 (1993). In other patents the reduction is performed using hydride metal salts such as NaBH₄ (DE 3441902, 1986) (EP 155591, 1985) (WO 9310068, 1993) (WO 2002/063703, 2007)). Finally, the selective reduction of the carbonyl group can be carried out through the Meerwein-Ponndorf-Verley (MPV) reaction using aluminium alcoholates as catalysts (WO 9732836, 1997). Thus, in the patent JP 55139330 (1980) the condensation between campholenal and butanal is performed using KOH in MeOH giving the α,β-unsaturated aldehyde which is purified and subsequently reduced with aluminium isopropoxide in the presence of isopropanol to Levosandal in a yield of 70%.

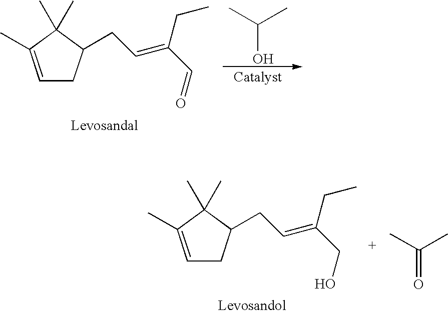

### SUMMARY OF THE INVENTION

In the present work we have surprisingly found that working with hydrated mixed oxides in the condensation between campholenal and butanal, the yield and selectivity to 2-ethyl-4-(2,2,3-trimethyl-3-cyclopenten-1-yl)-2-butenal is notably enhanced if a controlled amount of an aliphatic alcohol is added onto the catalyst previously to the reaction. Therefore, in this invention we present a new method for producing 2-ethyl-4-(2,2,3-trimethyl-3-cyclopenten-1-yl)-2-butenal in high yield and selectivity which involves the condensation of campholenal and butanal in the presence of a hydrated mixed oxide derived from LDH of divalent a trivalent metals in where a controlled amount of an aliphatic alcohol is added at the beginning of the reaction over the catalyst.

In accordance with the present invention, there is also provided a process for producing the unsaturated alcohol derivative of the 2-ethyl-4-(2,2,3-trimethyl-3-cyclopenten-1-yl)-2-butenal, this is Levosandol or Bacdanol (2-ethyl-4-(2,2,3-trimethyl-3-cyclopenten-1-yl)-2-buten-1-ol). Bacdanol is suitable as a synthetic perfume having a sandalwood-like scent.

In the present invention we present a process for producing levosandol starting from Levosandal using a heterogeneous bifunctional catalyst through the Meeerwein-Ponndorf-Verley reduction of Levosandal in the presence of a secondary alcohol as reducing agent. Moreover by coupling the condensation reaction between campholenal and butanal with the MPV reaction, we can obtain Levosandal through a one pot cascade process using the same bifunctional catalysts. The catalysts possess Lewis acid and basic sites, in such a way that it is able to catalyze as the first step, the aldol condensation between campholenal and butanal which yield the 2-ethyl-4-(2,2,3-trimethyl-3-cyclopenten-1-yl)-2-butenal. The aldehyde is reduced to the corresponding alcohol through the MPV reaction by the addition of a secondary alcohol in this case catalyzed by the Lewis acid and basic sites.

### DETAILED DESCRIPTION OF THE INVENTION

The present invention relates to a process for producing 2-ethyl-4-(2,2,3-trimethyl-3-cyclopenten-1-yl)-2-butenal useful as perfume or intermediate for perfume with high yield and selectivity by the cross-aldol condensation between campholenal and butanal in the presence of a controlled amount of an aliphatic alcohol using a bifunctional heterogeneous base catalysts, a process for producing Levosandol (2-ethyl-4-(2,2,3-trimethyl-3-cyclopenten-1-yl)-2-buten-1-ol) useful as perfume, starting from Levosandal through a MPV reduction using a bifunctional heterogeneous catalysts, as well as a process for producing levosandol (2-ethyl-4-(2,2,3-trimethyl-3-cyclopenten-1-yl)-2-buten-1-ol) through one pot cascade process which involves as the first step the aldol condensation between campholenal and butanal followed by the MPV reaction in the presence of a secondary alcohol and a bifunctional heterogeneous catalyst.

The synthesis of 2-ethyl-4-(2,2,3-trimethyl-3-cyclopenten-1-yl)-2-butenal is performed by mixing the reactants with a heterogeneous catalyst which has been treated previously by adding a controlled amount of an aliphatic alcohol. We have found that the addition of this controlled amount of the aliphatic alcohol is essential in order to obtain high conversion and selectivity to the desired compound.

The aliphatic alcohol can be selected between primary and secondary alcohols with one to 12 carbon atoms. The amount of aliphatic alcohol added is between 0.3 to 8 g of alcohol per gram of solid catalyst. Higher amounts of the aliphatic alcohol added over the catalyst showed to be detrimental for the reaction performance.

The condensation reaction between campholenal and butanal is performed preferentially using a molar ratio campholenal/butanal between 1:1 and 1:10.

The amount of catalyst can be between 1% and 40% wt with respect the total amount of reactants.

The use of solvent (except the amount of aliphatic alcohol required) in the above condensation is not necessary but optative. Also, the removal of water produced during the reaction is not particularly required.

The condensation can be carried out by mixing the reactants to react or by dropping the butanal over the mixture campholenal-catalyst slurry to gradually react with each of other.

The aldol condensation can be carried out under inert atmosphere or in the presence of air, in a batch reactor or in a fixed bed reactor.

In optimum reaction conditions, the procedure of the invention allows to obtain a conversion of campholenal higher 98% with selectivity to 2-ethyl-4-(2,2,3-trimethyl-3-cyclopenten-1-yl)-2-butenal higher than 95%.

The condensation reaction can be carried out in a continuous reactor or in a batch reactor. The reaction can be performed under inert atmosphere, at atmospheric pressure or in autoclave reactor at pressures between 1 and 20 atm and a temperature between 25 and 150° C., preferentially between 30 and 80° C.

Concerning to the reduction to Levosandal to Bacdanol through the Merweein-Ponndorf-Verley reaction the reaction is performed in the presence of a secondary alcohol as for instance isopropanol or 2-butanol which acts as reducing agent and as solvent. The molar ratio of secondary alcohol/Levosandal is between 10:1 to 50:1, preferentially between 20:1 and 30:1.

The ratio (in weight) of heterogeneous catalyst/Levosandal is between 1.6 to 0.5, preferentially between 1.0 to 0.8.

The reduction reaction can be carried out in continuous reactor or in a batch reactor. The reaction can be performed under inert atmosphere at atmospheric pressure or in autoclave reactor at pressures between 1 and 20 atm and a temperature between 25 and 150° C., preferentially between 60 and 90° C.

The bacdanol (2,2,3-trimethyl-3-cyclopenten-1-yl)-2-buten-1-ol) can be prepared through a one pot cascade process which involves as the first step the aldol condensation between campholenal and butanal in the presence of the heterogeneous catalysts. When the campholenal achieves preferentially above 90% of conversion, the reducing alcohol is added if necessary, and the reaction variables are adjusted for the MPV reaction occurs on the bifunctional heterogeneous catalyst giving bacdanol

The starting solid which refers the present invention which are used both in the first step condensation reaction and the second step MPV reduction are mixed oxides or hydrated mixed oxides derived of LDH of divalent and trivalent metals (M⁺²/N⁺³) of formula:

MₘNₙ(OH)₂ₘ₊₂ₙ(A)ₐbH₂O

Where M is a divalent metal cation selected between Mg⁺², Ni⁺², Zn⁺², Sn⁺², Fe⁺².

N is a trivalent metal cation selected between Al⁺³, Fe⁺³, Cr⁺³

A is a mono-, di- or trivalent anion which decomposes at temperatures between 300-700° C. giving a volatile gas.

m and n have values in such way that the ratio m/n is between 1 and 6.

b has a value between 1 and 10.

when A is an monovalent anion, a=n

when A is a divalent anion a=½

when A is a trivalent anion a=⅓

The layered double hydroxide precursor is calcined at temperatures between 300 and 700° C. and/or hydrated with an amount of water between 20-40 wt % previously to their use as catalyst.

As an example of these materials, the hydrotalcite, is aluminium magnesium hydroxide of laminar structure of formula Mg₆Al₂(OH)₁₆(CO₃)₂4H₂O which can be prepared with differed Al/Mg molar ratios. The calcinations of this material at temperatures above 450° C. leads to a Al—Mg mixed oxide (Mg₆Al₂(OH)₂).

The preparation of hydrotalcites are carried out starting from Mg(NO3)2, Al(NO3)3, Na2CO3 and NaOH of determined concentrations according to methodologies previously reported (for instance, J. Catal. 148 (1994) 205). The catalysts can be prepared means the aid of ultrasound radiation during the precipitation step according the methodology previously reported (Corma et al., J. Catal., 225 (2004) 316). At this point and for achieving the active operating catalyst claimed here it is required to add water in the range between 20 to 40 wt % with respect to the amount of the mixed oxide and an aliphatic alcohol between 1 and 12 carbon atoms in a ratio between 0.5 to 8 g/g of catalyst.

### Example I

The catalyst (0.5 g) (Al/Mg mixed oxide, with a molar ratio Mg/Al=3) was activated in situ previously to the reaction in a 10 mL two-necked flask reactor at 450° C. under N₂ flow during 6 h. After this time, the catalyst was hydrated by adding a 36 wt % of CO₂ free water and different amounts of MeOH were added. Then 17.5 mmol (1.26 g) of butanal and 6.8 mmol (1.03 g) of campholenal were added into the reactor which was equipped with a stirrer, and a reflux condenser. The resultant slurry was heated at 74° C. during 2 h under N₂ atmosphere. The conversion of campholenal and selectivity to Levosandal was dependent on the amount of MeOH added, as is showed in Table 1.

TABLE 1

| Methanol | Conversion | Yield | |
|---|---|---|---|
| (g/g catalyst) | Campholenal % | Levosandal % | Selectivity % |
| 0 | 55 | 41 | 74 |
| 1 | 75 | 76 | 98 |
| 3.4 | 66 | 63 | 95 |
| 8.0 | 59 | 44 | 75 |
| 12.0 | 50 | 36 | 73 |

### Example 2

The catalyst (0.5 g) (Al/Mg mixed oxide, with a molar ratio Mg/Al=3) was activated in situ previously to the reaction in a 10 mL two-necked flask reactor at 450° C. under N₂ flow during 6 h. After this time, the catalyst was hydrated by adding a 36% wt of CO₂ free water and different amounts of MeOH were added. Then 6.8 mmol (1.03 g) of campholenal were added into the reactor which was equipped with a dropping funnel charged with 17.5 mmol (1.26 g) of butanal, stirrer, and a reflux condenser. The resultant slurry was heated at 74° C. during 1.5 h under N₂ atmosphere, whereas the butanal was added at a rate of 0.8 mL/h. The conversion of campholenal and selectivity to Levosandal was dependent on the amount of MeOH added, as is showed in Table 2.

TABLE 2

| Methanol | Conversion | Yield | |
|---|---|---|---|
| (g/g catalyst) | Campholenal % | Levosandal % | Selectivity % |
| 0 | 75 | 60 | 80 |
| 0.3 | 88 | 75 | 85 |
| 0.6 | 95 | 85 | 89 |
| 1.7 | 98 | 90 | 92 |
| 3.4 | 80 | 77 | 96 |

### Example 3

The catalyst (0.5 g) (Al/Mg mixed oxide, with a molar ratio Mg/Al=3) was activated in situ previously to the reaction in a 10 mL two-necked flask reactor at 450° C. under N₂ flow during 6 h. After this time, the catalyst was hydrated by adding a 36% wt of CO₂ free water and then 160 wt % of 2-propanol was added. Then 6.8 mmol (1.03 g) of campholenal were added into the reactor which was equipped with a dropping funnel charged with 17.5 mmol (1.26 g) of butanal, stirrer, and a reflux condenser. The resultant slurry was heated at 74° C. during 2 h under N₂ atmosphere, whereas the butanal was added at a rate of 0.8 mL/h. The conversion of campholenal after 2 h reaction time was 90% with a selectivity to Levosandal of 95%.

### Example 4

The catalyst (1 g) of a Al/Mg mixed oxide, with a molar ratio Mg/Al=2 prepared by the conventional method as described in (J. Catal. 148 (1994) 205) was activated in situ previously to the reaction in a 10 mL two-necked flask reactor at 450° C. under air flow during 6 h and then under N₂ flow during 6 h. After this the reactor was charged with, 76 mmol (4.56 g) of isopropanol and 2 mmol (0.412 g) of Levosandal. The reactor was equipped with stirrer and a reflux condenser and heated at 90° C. under N₂ atmosphere. After 24 h reaction time a conversion of Levosandal of 90% with 70% selectivity to Bacdanol was obtained.

### Example 5

The catalyst (0.75 g) of a Al/Mg mixed oxide, with a molar ratio Mg/Al=2 prepared by sing ultrasound irradiation during the precipitation step as described by Corma et al., in J. Catal., 225 (2004) 316, was activated in situ previously to the reaction in a 10 mL two-necked flask reactor at 450° C. under air flow during 6 h and then under N₂ flow during 6 h. After this the reactor was charged with, 76 mmol (4.56 g) of isopropanol and 2 mmol (0.412 g) of Levosandal. The reactor was equipped with stirrer and a reflux condenser and heated at 90° C. under N₂ atmosphere. After 4 h reaction time a conversion of Levosandal of 99% with 70% selectivity to Bacdanol was obtained.

We claim:

1. A process for producing 2-ethyl-4-(2,2,3-trimethyl-3-cyclopenten-1-yl)-2-butenal (Levosandal) by a cross-aldol condensation between campholenal and butanal in the presence of a controlled amount of an aliphatic alcohol using heterogeneous bifunctional catalysts.

2. The process of claim 1 wherein the heterogeneous catalysts are selected from the group consisting of mixed oxides or hydrated mixed oxides derived of layered double hydroxides (LDH) of divalent and trivalent metals (M⁺²/N⁺³) of formula:
MₘNₙ(OH)₂ₘ₊₂ₙ(A)ₐbH₂O
Where M is a divalent metal cation selected from the group consisting of Mg⁺², Ni⁺², Zn⁺², Sn⁺², Fe⁺²;
N is a trivalent metal cation selected from the group consisting of Al⁺³, Fe⁺³, Cr⁺³;
A is a mono-, di- or trivalent anion which decomposes at temperatures between 300-700° C. giving a volatile gas;
m and n have values wherein the ratio m/n is between 1 and 6;
b has a value between 1 and 10.
when A is an monovalent anion, a=n
when A is a divalent anion a=½
when A is a trivalent anion a=⅓

4. The process of claim 2 wherein the catalyst is a mixed oxide selected from a heterogeneous mixed oxides comprising divalent and trivalent metals with a molar ratio M⁺²/M⁺³ between 1 and 6.

5. The process of claim 2 wherein the catalyst is a mixed oxide selected from a heterogeneous mixed oxides comprising aluminium and magnesium derived from hydrotalcites with a molar ratio Mg/Al between 1 and 6.

6. The process of claim 2 wherein the catalyst is a mixed oxide which has been submitted to a calcination process at temperatures between 40-700° C.

7. The process of claim 2 wherein the catalyst has been submitted to a partial hydration process by adding between 20 to 40% of water with respect to the amount of the mixed oxide.

8. The process of claim 1 wherein the synthesis of 2-ethyl-4-(2,2,3-trimethyl-3-cyclopenten-1-yl)-2-butenal is performed by mixing the reactants with a heterogeneous catalyst which has been treated previously by adding a controlled amount of an aliphatic alcohol.

9. The process of claim 1 wherein the aliphatic alcohol can be selected from the group consisting of primary and secondary alcohols with 1 to 12 carbon atoms.

10. The process of claim 1 wherein the amount of aliphatic alcohol added is between 0.3 to 8 g of alcohol per gram of catalyst.

11. The process of claim 1 wherein the reaction between campholenal and butanal is performed using a molar ratio campholenal/butanal of 1:1 to 1:10.

12. The process of claim 1 wherein the amount of catalyst can be between 1 and 40% with respect the total amount of reactants.

13. The process of claim 1 wherein the condensation can be carried out by mixing the reactants to react with each other or by dropping the butanal over the mixture campholenal-catalyst slurry to gradually react with each of other.

14. The process of claim 1 wherein the aldol condensation is carried out under inert atmosphere, at atmospheric pressure or in autoclave reactor at pressures between 1 and 20 atm and a temperature between 25 and 150° C.

15. The process of claim 1 where 2-ethyl-4-(2,2,3-trimethyl-3-cyclopenten-1-yl)-2-buten-1-ol can be produced through one pot cascade process wherein the first step is the aldol condensation between campholenal and butanal followed by an MPV reaction.

16. A process for producing levosandol (2-ethyl-4-(2,2,3-trimethyl-3-cyclopenten-1-yl)-2-buten-1-ol), starting from 2-ethyl-4-(2,2,3-trimethyl-3-cyclopenten-1-yl)-2-butenal through a MPV reduction using a bifunctional heterogeneous catalysts.

17. The process of claim 16 wherein the heterogeneous catalysts are selected from the group consisting of mixed oxides or hydrated mixed oxides derived of layered double hydroxides (LDH) of divalent and trivalent metals (M⁺²/N⁺³) of formula:
MₘNₙ(OH)₂ₘ₊₂ₙ(A)ₐbH₂O
Where M is a divalent metal cation selected from the group consisting of Mg⁺², Ni⁺², Zn⁺², Sn⁺², Fe⁺²;
N is a trivalent metal cation selected from the group consisting of Al⁺³, Fe⁺³, Cr⁺³;
A is a mono-, di- or trivalent anion which decomposes at temperatures between 300-700° C. giving a volatile gas;
m and n have values wherein the ratio m/n is between 1 and 6;
b has a value between 1 and 10.
when A is an monovalent anion, a=n
when A is a divalent anion a=½
when A is a trivalent anion a=⅓

18. The process of claim 16 wherein the catalyst is a mixed oxide selected from a heterogeneous mixed oxides comprising divalent and trivalent metals with a molar ratio M⁺²/M⁺³ between 1 and 6.

19. The process of claim 16 wherein the catalyst is a mixed oxide selected from a heterogeneous mixed oxides comprising aluminium and magnesium derived from hydrotalcites with a molar ratio Mg/Al between 1 and 6.

20. The process of claim 16 wherein the catalyst is a mixed oxide which has been submitted to a calcination process at temperatures between 40-700° C.

21. The process of claim 16 wherein the catalyst has been submitted to a partial hydration process by adding between 20 to 40% of water with respect to the amount of the mixed oxide.

22. The process of claim 16 wherein the synthesis of 2-ethyl-4-(2,2,3-trimethyl-3-cyclopenten-1-yl)-2-butenal is performed by mixing the reactants with a heterogeneous catalyst which has been treated previously by adding a controlled amount of an aliphatic alcohol.

23. The process of claim 16 wherein the aliphatic alcohol can be selected from the group consisting of primary and secondary alcohols with 1 to 12 carbon atoms.

24. The process of claim 16 wherein the amount of aliphatic alcohol added is between 0.3 to 8 g of alcohol per gram of catalyst.

25. The process of claim 16 wherein the reaction between campholenal and butanal is performed using a molar ratio campholenal/butanal of 1:1 to 1:10.

26. The process of claim 16 wherein the amount of catalyst can be between 1 and 40% with respect the total amount of reactants.

27. The process of claim 16 wherein the condensation can be carried out by mixing the reactants to react with each other or by dropping the butanal over the mixture campholenal-catalyst slurry to gradually react with each of other.

28. The process of claim 16 wherein the aldol condensation is carried out under inert atmosphere, at atmospheric pressure or in autoclave reactor at pressures between 1 and 20 atm and a temperature between 25 and 150° C.

29. The process of claim 16 wherein the reduction of 2-ethyl-4-(2,2,3-trimethyl-3-cyclopenten-1-yl)-2-butenal is performed in the presence of a secondary alcohol as for instance isopropanol or 2-butanol.

30. The process of claim 29 wherein the molar ratio between the secondary alcohol and 2-ethyl-4-(2,2,3-trimethyl-3-cyclopenten-1-yl)-2-butenal is between 10:1 to 50:1.

31. The process of claim 30 wherein the ratio (in weight) of the heterogeneous catalyst and 2-ethyl-4-(2,2,3-trimethyl-3-cyclopenten-1-yl)-2-butenal is between 1.6 to 0.5.

32. The process of claim 16 where the reduction reaction can be carried out in continuous reactor or in a batch reactor under inert atmosphere at atmospheric pressure or in autoclave reactor at pressures between 1 and 20 atm and a temperature between 25 and 150° C.

33. The process of claim 16 where 2-ethyl-4-(2,2,3-trimethyl-3-cyclopenten-1-yl)-2-buten-1-ol can be produced through one pot cascade process wherein the first step is the aldol condensation between campholenal and butanal followed by an MPV reaction.
